(19) United States
(12) Patent Application Publication
(10) Pub. No.: US 2023/0149147 A1
(43) Pub. Date: May 18, 2023

(54) SURGICAL MESH IMPLANT FOR HERNIA REPAIR AND METHODS OF USE

(71) Applicant: American University of Beirut, Beirut (LB)

(72) Inventors: Ramzi Alami, Beirut (LB); Ali Tehrani, Aalto (FI); Marwan El Sabban, Beirut (LB)

(21) Appl. No.: 17/983,611

(22) Filed: Nov. 9, 2022

Related U.S. Application Data
(60) Provisional application No. 63/277,237, filed on Nov. 9, 2021.

(51) Int. Cl. A61F 2/00

(52) U.S. Cl. CPC A61F 2/0063; A61F 2210/0004; A61F 2002/009; A61F 2002/0081; A61F 2240/001

(57) ABSTRACT

A mesh implant is disclosed and comprises a single sheet, highly porous, adhesion-resistant, tensile surgical implant composed of a gradually biodegradable synthetic polymer material that is electrospun into nanofibers and randomly stacked to form a three-dimensional (3D) mesh. The mesh implant is for tissue repair and hernia repair. The single-sheet design reduces the foreign material that make up the mesh implant, which minimizes mesh implant rejection. The gradually biodegradable nature of the mesh implant guarantees that the mesh stays in place and supports the repaired site long enough until a proper scar tissue has built up, after which the mesh implant disappears from the body, therefore preventing pain and irritability. The 3D design of the nanofibrous network and the high porosity of the mesh implant facilitate cell attachment, infiltration, and proliferation, all necessary for scar tissue formation, mesh integration, wound healing, and proper defect closure.

### CROSS-REFERENCE TO RELATED APPLICATIONS

The present application claims priority to U.S. Patent Application No. 63/277,237, filed Nov. 9, 2021, herein incorporated by reference in its entirety.

### BACKGROUND

The invention generally relates to advanced polymeric materials, regenerative medicine and general surgery. More specifically, the invention relates to a surgical prosthesis in the form of a flat nanofibrous three-dimensional mesh that is used in tissue repair, namely hernia repair.

Hernias are protrusions or projections of organs through the wall of the cavity where they are normally contained. If left untreated, hernias may become obstructed and difficult to restore to a desirable condition, which may result in a potentially fatal state. About 20 million hernia repair procedures are performed annually around the world. Standard treatment of hernia involves the insertion of a synthetic surgical mesh to reinforce the compromised wall and support the protruding organ. The majority of the surgical meshes on the market are permanent, non-biodegradable synthetic polymers i.e. they stay in place for the rest of the person's life. Although non-biodegradable meshes decrease the chance of hernia recurrence, they may cause major discomfort, movement restriction and chronic pain.

The alternative to non-biodegradable meshes is the use of synthetic or biological biodegradable meshes that gradually degrade in vivo to form a native and remodeled tissue at the surgery site. As biodegradable meshes eventually disappear from the body, they do not cause long-term irritation that could lead to chronic pain and limited mobility. However, the use of biodegradable meshes is primarily limited by the possibility of hernia recurrence after the mesh has degraded. Following the hernia procedure, the repaired site takes about three to six months to build up scar tissue; the latter which strengthens and supports the compromised region. Biodegradable meshes must provide temporary support to the repaired site until the scar tissue is strong enough to assume this role. Therefore, biodegradable meshes are expected to remain in place for at least six months while the scar tissue is building. However, the majority of biodegradable meshes have a high rate of degradation, and they disappear from the body well before the six-month mark. During this period, the scar tissue is not developed enough to support the repaired site. As a result, the use of biodegradable meshes is associated with a high incidence of hernia recurrence.

Many modern biodegradable meshes are designed to be multilayered to overcome some of the drawbacks of traditional biodegradable meshes. For instance, some meshes are composed of a rapidly degrading layer that triggers the inflammatory response necessary for wound healing and a slowly degrading layer that supports the tissue. Other meshes are designed to have an additional layer that prevents tissue adhesion. While these designs serve their intended purpose, multilayered meshes have a higher weight compared to single-sheet meshes. As a result, they pose a higher risk for connective tissue irritation and mesh rejection.

Finally, biodegradable meshes have limited availability (in case of synthetic meshes), high cost (in case of biological meshes), restriction in mesh size and shape, and most of them have a two-dimensional (2D) structure. 2D meshes lack the structural network necessary for cell attachment, infiltration, and proliferation. As the infiltration of white blood cells and fibroblasts is crucial for hernia defect closure, the use of 2D meshes in hernia repair is often accompanied with poor wound healing and delayed defect closure.

The present invention attempts to solve these problems as well as others.

### SUMMARY OF THE INVENTION

The mesh implant comprises a flat, single-sheet, highly porous, adhesion-resistant, and tensile surgical implant composed of a slowly biodegradable synthetic polymer that is electrospun into nanofibers and randomly stacked to form a three-dimensional (3D) mesh.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a cross section of Scanning Electron Microscopy (SEM) image showing a flat single-sheet nanofibrous three-dimensional mesh with average thickness in the order of 200 μm, according to one embodiment.

FIG. 2 is a top view of a photograph of the flat single-sheet nanofibrous three-dimensional mesh of Example 1 made from a biocompatible and slowly biodegradable polymer. This mesh has a single-sheet nanofibrous three-dimensional structure, and it is manufactured using electrospinning.

FIG. 3 is a magnified SEM image that shows an up-close view of the nanofibers of the mesh and the large pores between them, according to one embodiment.

FIG. 4 is an SEM image showing the surface morphology and a highly porous nanofibrous three-dimensional mesh, according to one embodiment.

FIG. 5 is a schematic diagram of one embodiment of the electrospinning machine that can be used in the manufacturing of the mesh implant where (A) is the syringe pump and conductive nozzle, (B) is the high voltage power supply, and (C) is a rotating drum.

FIG. 6 is a photograph of the electrospinning machine to manufacture one embodiment of the mesh implant as described in Example 1.

FIG. 7 is an SEM image of the mesh implant of example 1 as embedded with fibroblasts for a period of maximum 14 days. On day 14, the mesh was imaged using scanning electron microscopy. FIG. 7 shows the attachment and proliferation of fibroblasts (nodule-like structures) on the surface of the mesh (fibrous structures). The fibroblasts completely cover the surface of the mesh.

FIG. 8 is a confocal microscopy image of the mesh implant of Example 1 that was embedded with fibroblasts for 14 days. This embodiment has a thickness in the order of about 200 and the image shows that on day 14, the fibroblasts (blue-colored dots) had infiltrated the mesh (green-colored fibers), and the fibroblasts spread across the whole structure of the mesh.

FIG. 9 is a graph of the mesh implant of Example 1 that was embedded with fibroblasts for a period of maximum 90 days. At different increasing time points, a sample of the mesh was collected and its longitudinal and transverse tensile strengths were measured. The line graph in this figure details the change of the percentage (%) of retained longitudinal and transverse tensile strength of the embodiment of the mesh implant as function of the time (in days) this embodiment was embedded with fibroblasts. The results show that the percentage of retained longitudinal and transverse tensile strengths of the mesh decreases the longer the mesh is embedded with the fibroblasts.

FIG. 10A is a histological image (20×) of an H&E-stained tissue section collected from the repaired site of a ventral hernia that was created in a pig model and repaired using the mesh implant of Example 1. The mesh was placed directly over the repaired site. Animal sacrifice and tissue collection took place one month following the hernia procedure. The image shows the mesh nanofibers (pinkish-white fibrous structures) present in the tissue section. The mesh nanofibers are present in abundance in the tissue sample indicating minimum degradation. The mesh is well integrated into the neighboring tissues as evidenced by its infiltration with white blood cells and fibroblasts (purple-colored structures) and the lack of signs of necrosis. Imaging was carried out using light microscopy.

FIG. 10B is a histological image (20×) of an H&E-stained tissue section collected from the repaired site of a ventral hernia that was created in a pig model and repaired using the mesh implant of Example 1. The mesh was placed directly over the repaired site. Animal sacrifice and tissue collection took place two months following the hernia procedure. The image shows the mesh nanofibers (pinkish-white fibrous structures) present in the tissue section. The image also shows signs of biodegradation as evidenced by the lesser number of visible nanofibers compared to FIG. 10A. The mesh is well integrated into the neighboring tissues as evidenced by its infiltration with white blood cells and fibroblasts (purple-colored structures) and the lack of signs of necrosis. Imaging was carried out using light microscopy.

FIG. 10C is a histological image (20×) of an H&E-stained tissue section collected from the repaired site of a ventral hernia that was created in a pig model and repaired using the mesh implant of Example 1. The mesh was placed directly over the repaired site. Animal sacrifice and tissue collection took place six months following the hernia procedure. The image shows the mesh nanofibers (pinkish-white fibrous structures) present in the tissue section. The image also shows continued biodegradation as evidenced by the reduced number of visible nanofibers left in the tissue section compared with FIG. 10B. The mesh is well integrated into the neighboring tissues as evidenced by its infiltration with white blood cells and fibroblasts (purple-colored structures) and the lack of signs of necrosis. Imaging was carried out using light microscopy.

FIG. 11 is a histological image (20×) of an H&E-stained tissue section collected from the repaired site of a ventral hernia that was created in a mouse model and repaired using the mesh implant of Example 1. The mesh was placed directly over the repaired site. Animal sacrifice and tissue collection took place one month following the hernia procedure. The image represents three stages of mesh infiltration by white blood cells and fibroblasts. Zone I of the tissue represents the area of the mesh (pinkish-white fibrous structures) that has been fully infiltrated with cells (purple-colored structures). Zone II represents the area of the mesh where cell infiltration is in progress. Numerous cells (purple-colored structures) can be seen spread across the whole area of the mesh (pinkish-white fibrous structures). Zone III represents the area of the mesh where only fibers can be seen indicating that cell infiltration has not reached this area yet.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Description of Embodiments

Generally speaking, the mesh implant is a flat, single sheet, highly porous, adhesion-resistant, and tensile surgical implant composed of a slowly biodegradable synthetic polymer that is electrospun into nanofibers and randomly stacked to form a three-dimensional (3D) mesh. The mesh implant is suitable for tissue repair, namely hernia repair. In one embodiment, the use of the mesh implant in hernia repair. However, in other embodiments, the use of the mesh implant is not limited to hernia, as it is used in other procedures such as, but not limited to, wound healing, repair of injuries to the bone tissue, nerve tissue, muscle tissue or skin tissue, and the treatment of burn injuries. Hernias repaired by the mesh implant include, but are not limited to, inguinal, femoral, umbilical, incisional, epigastric, and hiatal. The mesh implant is placed on the defect on the uncovered fascia. The mesh implant helps overcome the common drawbacks of traditional surgical meshes utilized in hernia repair.

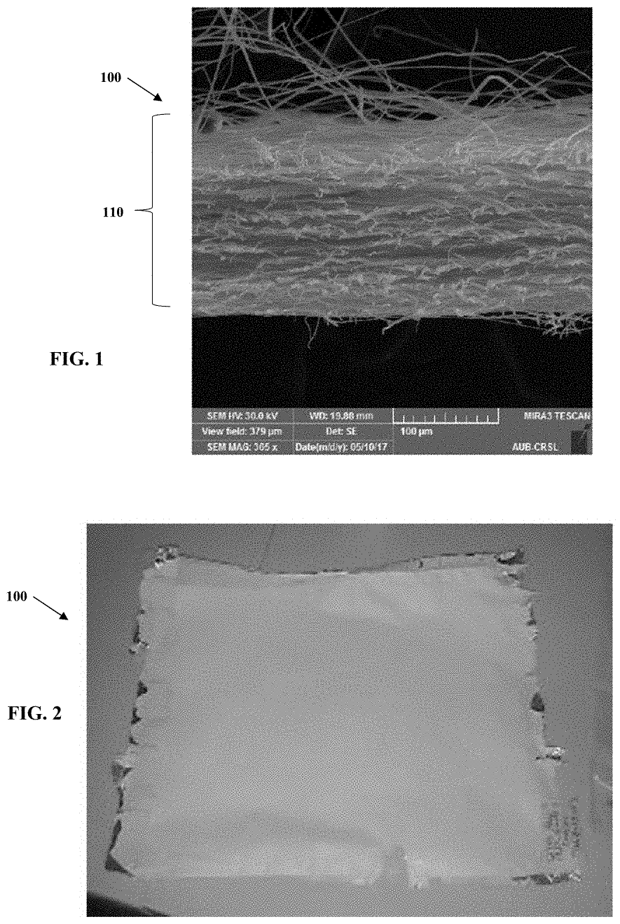

As shown in FIG. 1, the mesh implant 100 comprises a single layer 110 of polymeric material. The single-sheet 110 mesh incorporates the minimal amount of foreign material necessary for the function of the mesh implant 100, making the latter a light-weight implant 100. As a result, the mesh implant 100 causes no damage to the surrounding connective tissue following its placement on the hernia defect, which facilitates mesh implant integration and defect closure. In contrast, multilayered meshes incorporate an extra amount of foreign material in their make-up, which trigger a severe inflammatory reaction and lead to mesh rejection. In addition, the single-sheet structure of this mesh confers flexibility to the implant, which facilitates its placement firmly on the repaired site, prevents it dislocation, and allows it to adapt to the movements of the body.

The mesh implant 100 comprises a gradually biodegradable synthetic polymer material, wherein the polymer material may be a single polymer, copolymers, polymer blends or various polymer parts. The polymer is biocompatible, gradually biodegradable, and is electrospun into a single layer. For one embodiment, the degradation of the polymeric material is at least six months, which allows the mesh implant to support the repaired site for at least six months while a scar tissue is building. The placement of the mesh implant on the repaired site triggers the formation and buildup of a scar tissue as a response to the presence of mesh's foreign material. The scar tissue is formed of a fibrotic tissue that takes about three to about six weeks following hernia procedure to fully form and strengthen, after which the fibrotic tissue can replace the mesh and support the repaired site on its own. Therefore, following its placement on the hernia defect, the degradation of the mesh implant is slow enough to allow the implant to stay in place for at least six months until the scar tissue is strong enough to support the repaired site all on its own. Most available biodegradable meshes disintegrate very quickly following hernia procedure while the scar tissue has not fully developed, which leaves the repaired site without physical support. As a result, the use of biodegradable meshes in hernia repair is limited by the high risk of hernia recurrence. The mesh implant decreases the risk of hernia recurrence by ensuring that the repaired site is fully supported when the scar tissue is still not strong enough to assume this role. After the six-month mark, the mesh implant continues to degrade until it eventually disappears from the body thereby eliminating the risk of long-term foreign pain and irritability which are usually encountered when non-biodegradable meshes are used in hernia repair.

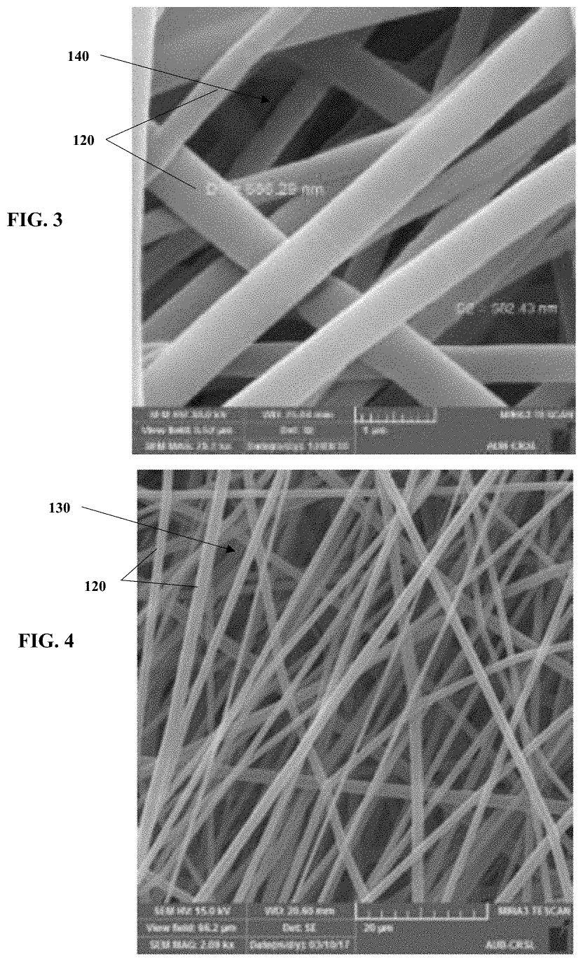

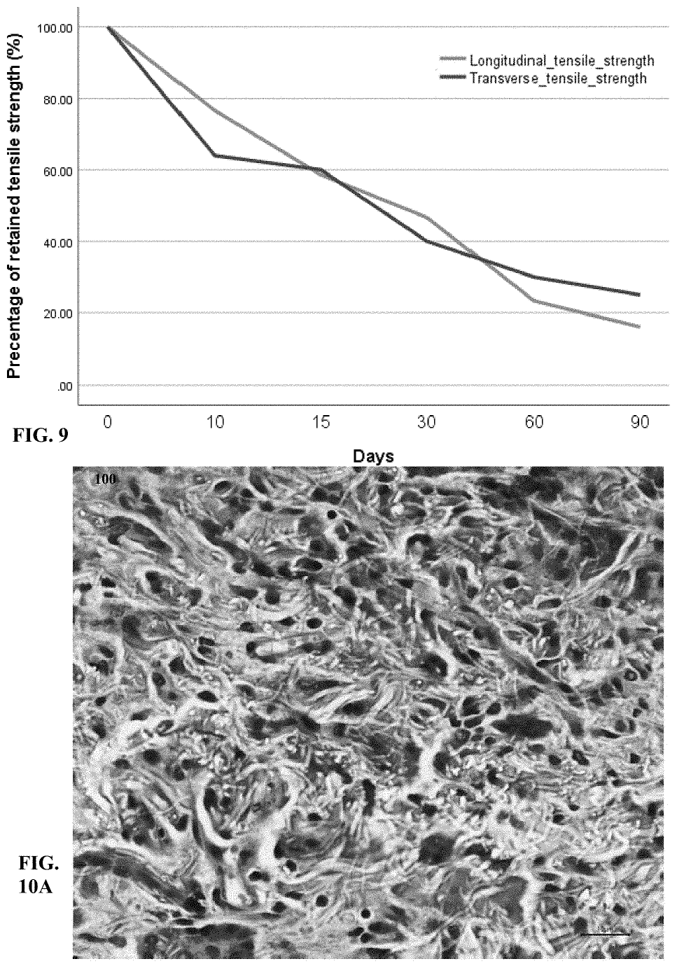

In one embodiment, the mesh implant provides physical support to the repaired hernia site and reduce the risk of hernia recurrence. The mesh implant is strong enough to withstand the pressure exerted by the internal organs of the patient on the compromised abdominal wall and the mesh itself without breaking. Two elements control the mesh implant's strength: its thickness and tensile strength. The mesh implant of one embodiment has a thickness less than about 75 μm, as shown FIG. 1, with other embodiments with thickness of about 200 μm showing favorable results in terms of strength and physical support. Accordingly, the fibers 120 of this mesh implant have diameters that are about 0.2 μm or higher, as shown in FIG. 3. Next, any mesh implant to be used in hernia repair has a uniaxial tensile strength of at least 16 N/cm in order to counter and withstand the pressure exerted by the internal organs on the abdominal wall during the daily activities of the patient. Both longitudinal and transverse tensile strengths of the mesh of this embodiment are higher than 16 N/cm, while ensuring that the flexibility of the mesh is maintained. Once the mesh implant is placed in a biological environment, the mesh implant losses about 20% of the original longitudinal tensile strength by the 10^{th} day and about 35% of the original transverse tensile strength by the 10^{th} day, which indicates a gradual biodegradability, as shown in FIG. 9. By 15 days, the mesh implant losses about 40% of its original longitudinal tensile strength and about 40% of its original transverse tensile strength. By 30 days, the mesh implant losses about 50% of its original longitudinal tensile strength and about 60% of its original transverse tensile strength. By 60 days, the mesh implant losses about 75% of its original longitudinal tensile strength and about 70% of its original transverse tensile strength. By 90 days, the mesh implant losses about 84% of its original longitudinal tensile strength and about 75% of its original transverse tensile strength. This loss of its original longitudinal tensile strength and original transverse tensile strength indicate that as the mesh implant when placed in a biological environment, the fibers gradually degrade which explains the loss of their mechanical strength.

In addition to physically supporting the injured wall, the mesh implant facilitates wound healing at the repaired hernia site leading to a successful defect closure. Wound healing requires the infiltration of white blood cells and fibroblasts into the repaired site in order to initiate the injury repair and promote tissue ingrowth. The mesh implant promotes and facilitates cell attachment, infiltration, proliferation and differentiation. Using electrospinning, this mesh implant comprises a highly porous nanofibrous three-dimensional (3D) structure 140. The gradually degradable polymer is electrospun into nanofibers 120, which are randomly distributed and stacked to form a flat 3D mesh 140, as shown in FIG. 4. The nanofibrous 3D structure 140 of this mesh has a high surface area to volume ratio and mimics the structure of the extracellular matrix (ECM), which promote cell attachment, infiltration, proliferation, and differentiation on the nanofibers. Furthermore, the mesh implant comprises a highly porous structure with the pores 140 wide enough to allow the infiltration of white blood cells and fibroblasts, so that the cells have access to the nanofibers where they attach, proliferate, and differentiate. The average number of pores of this mesh is about 70 pores/cm², and the pore size is greater than about 2 μm while ensuring that the tensile strength of the mesh is maintained, as shown in FIG. 4. The mesh implant solves the problem that is usually encountered with 2D meshes. The structure of 2D meshes does not facilitate the adhesion and infiltration of cells, causing the use of such meshes in hernia repair to be associated with a high risk of poor wound healing and disrupted defect closure. The highly porous structure of the mesh implant overcomes the problem of poor cell infiltration that is usually encountered with other commonly available meshes that have low porosity and small pore size. The mesh implant represents a scaffold for growing isolated differentiable cells, most notably stem cells and progenitor cells. Examples of differentiable cells that can be grown on the mesh implant include, but are not limited to, stem or progenitor cells of the blood, cartilage, bones, skin, and nerves. Therefore, in addition to its role in hernia repair, the mesh implant can be used in the repair of tissue injury such as, but not limited to, wound healing, repair of injuries to the bone, nerves, or skin, and treatment of burn injuries.

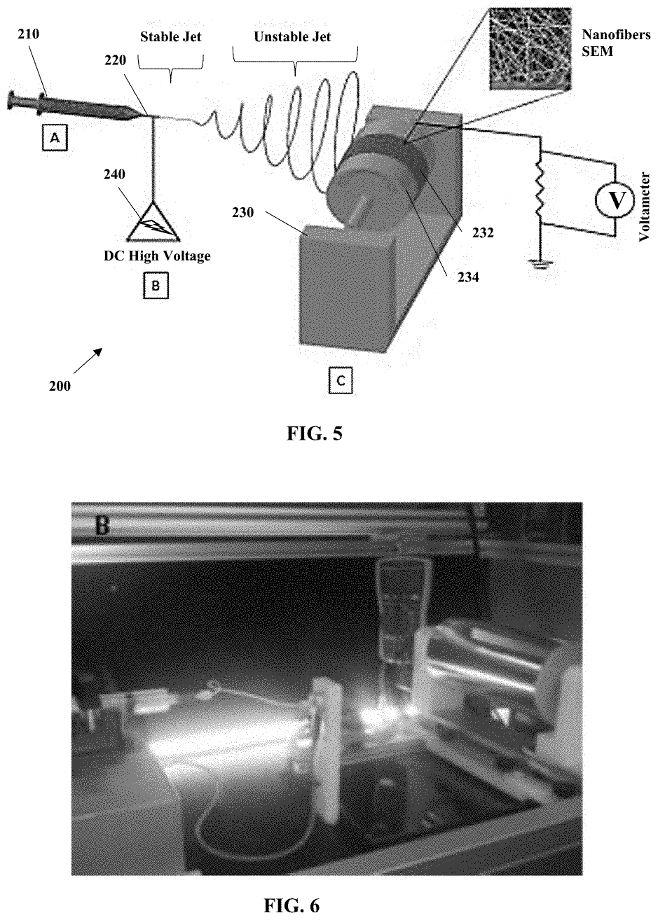

The mesh implant is manufactured by electrospinning to achieve a single-sheet highly porous nanofibrous 3D flat structure. For one embodiment, the slowly biodegradable synthetic polymer material that is used in the manufacturing of this mesh implant is suitable for electrospinning. The electrospinning machine 200 comprises of a syringe pump 210, a conductive nozzle 220, a rotating drum 230, and a high voltage power supply 240 connected to the nozzle 220, as shown in FIG. 5. The rotating drum 230 is covered by aluminum foil 232 and a commercial polyester screen fabric 234. For this embodiment, the electrospinning parameters are selected such that the manufactured mesh implant has maximized thickness and pore size. The maximized thickness provides maximum physical support to the repaired hernia site, while the maximized pore size guarantees maximum cell attachment and infiltration which facilitates wound healing and proper defect closure. Finally, the single-sheet electrospun nanofibrous 3D structure of this mesh implant provides for an adhesion-resistant implant. This mesh implant is an upgrade from the commercially available 2D meshes, since electrospinning offers the liberty of manufacturing tailor-made surgical meshes with customizable shapes, sizes and mechanical properties that cater to the market needs. In one embodiment, other manufacturing techniques often yield knitted or woven meshes that are two-dimensional, which presents restrictions in mesh size and shape. In another embodiment, electrospinning is a low-cost manufacturing technique with readily available equipment, making the mesh of this mesh implant an affordable product, unlike other available meshes, such as biological meshes. Finally, the most favorable feature of electrospinning is its scalable productivity. In case of growing market demand, electrospinning makes it possible to increase the production of the mesh implant without major increase in costs. The simplicity of the design of the electrospinning machine makes it possible to control parameters that will result in increase of the number of manufactured meshes per unit of time. Therefore, the electrospun nature of the mesh implant makes an affordable, yet profitable, product.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Fabrication of a Single-Sheet Highly Porous Gradually Biodegradable Nanofibrous Three-Dimensional Mesh by Electrospinning

One embodiment of the mesh implant was manufactured using an FDA-approved gradually biodegradable polymer. A laboratory-scale electrospinning machine (FLUIDNATEK LE-10, BIOINICIA, Spain) was used for the fabrication of this embodiment. The machine 200 comprises a syringe pump 210, a conductive nozzle 220 with an internal diameter of more than 50 mm and an outer diameter of less than 1.2 mm, a rotating drum 230 with a diameter of 10 cm, and a high voltage power supply 240 connected to the conductive nozzle 220. The rotating drum was covered by aluminum foil and a commercial polyester screen fabric, as shown in FIG. 6.

The polymer was dissolved in a mixture of Tetrahydrofuran and dimethylformamide by magnet stirring the mixture. The electrospinning parameters such as the applied voltage (V), PLDL initial concentration, polymer solution feed rate (FR), tip to collector distance (TCD), and collector rotational speed (CS) were selected such that the resultant mesh had maximized thickness and pore size. To that end, the mesh implant was electrospun under low applied voltage and low CS. The lab temperature and relative humidity were 25±1° C. and 35±5%, respectively. The syringe pump was used to feed the polymer solution to the tip of the spinning nozzle. Under these conditions, the polymer was electrospun into nanofibers, which were randomly distributed and stacked on the rotating drum to give a nanofibrous three-dimensional mesh, as shown in FIG. 2.

### Example 2: Analyzing the Morphology and Diameter of the Mesh Fibers

The morphology and diameter of the electrospun fibers of the mesh implant of Example 1 was viewed and analyzed by a Scanning Electron Microscope (SEM) (MIRA 3 LMU Tescan, Czech Republic). Several samples (1×1 cm) from different parts of the mesh were cut and fixed on aluminum holders by carbon adhesive tabs. The samples were coated with about a 20 nm layer of gold using Q150 Sputter Coater (Quorum Technologies) under a low current. The sample holders were then placed in the vacuum chamber of the SEM for imaging under scanning electron (SE) mode detector. The average fiber diameter of each sample was determined by averaging over at least 100 fiber measurements and their distribution was analyzed and plotted by OriginLab. Analysis of all samples showed a uniform surface where the fibers are randomly oriented. Moreover, all samples were bead free. A view of the samples using larger magnification was used for determining the average fiber diameter and the fiber diameter distribution.

### Example 3: Determining the Pore Size of the Mesh

The size of the pores of the mesh of Example 1 was measured by Capillary Flow Porometer (PMI-1100, NY, USA) by taking four samples from different parts of the mesh. Each sample mesh (˜5 cm×˜5 cm) was placed between the sample holders and wetted with a low surface tension liquid, Galwick, to spontaneously fill the pores. The average pore size of each sample was calculated from Eq. 1:

ΔP=4γβ cos θ/D; (Eq. 1)

where ΔP is the change in pressure needed to open the pore, D is the pore size, γ is the wetting agent surface tension, θ is the contact angle, and β is the pore geometrical correction factor (β≅0.71).

### Example 4: Visualization of Cell Attachment to the Mesh

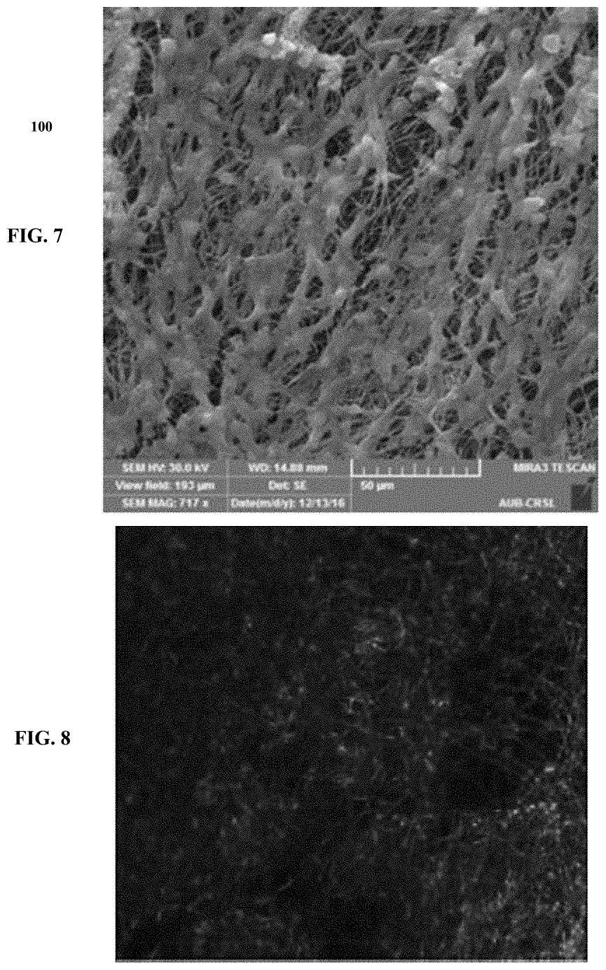

Fibroblasts were maintained in a humidified atmosphere at about 37° C. and about 5% CO2, refreshing medium every two days. They were grown in Dulbecco's Modified Eagle Medium AQ (DMEM AQ) media supplemented with 10% fetal bovine serum (FBS) and 1% penicillin-streptomycin. Samples of the mesh of Example 1 were cut in 6×2 cm rectangles and seeded with fibroblasts of concentration 20×104 cells/cm² for a period of maximum 14 days. After incubation, the mesh samples were washed with PBS twice and then gently fixed with 2.5% glutaraldehyde at 4° C. for 60 minutes. The samples were then rinsed three times with PBS and two times with distilled water. Each sample was then dehydrated with a graded series of ethanol (25%, 50%, 75%, 95%, and 100%) for 5 minutes. After drying, they were coated with a thin layer of gold using Q150 Sputter Coater (Quorum Technologies) and visualized by SEM. SEM micrographs revealed that, by day 14, fibroblasts had attached to the mesh and almost wholly covered the whole surface of the implant indicating that cells proliferated in time, as shown in FIG. 7. These results indicate that the mesh implant promotes cell attachment which is important for proper wound healing.

### Example 5: Visualization of Cell Infiltration of the Mesh Implant

Fibroblasts were maintained in a humidified atmosphere at 37° C. and 5% CO2, refreshing medium every two days. They were grown in Dulbecco's Modified Eagle Medium AQ (DMEM AQ) media supplemented with 10% fetal bovine serum (FBS) and 1% penicillin-streptomycin. Samples of the mesh of Example 1 were cut in 6 cm×2 cm rectangles and seeded with fibroblasts of concentration 50×103 cells/cm² for a period of maximum 14 days. A laser scanning confocal microscope was used to visualize the cell infiltration into the mesh at different time points following the seeding. For better visualization of mesh, a solvent color (quinizarin) was added to the polymeric solution before electrospinning. The resulting mesh appeared in green color by the FITC filter. To examine cell infiltration, fibroblasts seeded on the nanofibrous mesh were fixed with paraformaldehyde (PFA), washed with phosphate buffer saline (PBS), and stained with Hoechst 33342 (Eugene, USA) at 1 μg/ml for 10 minutes at room temperature to visualize the cell nuclei. The samples were then washed with PBS, mounted on slides using Prolong Anti-fade kit and observed by microscopy. Z-stacks of images were acquired using a 63×/1.46 Oil Plan Apochromatic objective. The results showed that, by day 14, fibroblasts had proliferated forming a confluent layer. Moreover, Z stack 3D images show that cells were able to infiltrate the mesh in depth. Finally, cells were shown to be widely spread across the mesh and not concentrated at a specific position, as shown in FIG. 8. These results indicate that the mesh of this mesh implant promotes cell attachment, infiltration and proliferation which are important for proper wound healing.

### Example 6: Testing the Uniaxial Tensile Stress of the Mesh Implant

a. Uniaxial Tensile Strength of Mesh Implant of Example 1

Uniaxial tensile stress testing of the dry mesh implant was performed using the tensile testing machine (Instron 5943, USA) according to the ASTM D882-10 standard test method. Samples of the mesh of Example 1 were carefully cut into rectangular forms of 2 cm width and 6 cm length. Tensile testing was done at an extension rate of 5 mm/min. The gauge distance between the grippers was modified to 4 cm, and the width was modified to 2 cm. The results showed that both the longitudinal and transverse tensile strengths of the mesh of example 1 were greater than 16 N/cm.

b. Uniaxial Strength of the Mesh Implant Embedded with Mouse Fibroblasts

Fibroblasts were maintained in a humidified atmosphere at 37° C. and 5% CO2, refreshing medium every two days. They were grown in Dulbecco's Modified Eagle Medium AQ (DMEM AQ) media supplemented with 10% fetal bovine serum (FBS) and 1% penicillin-streptomycin. Samples of the mesh implant of Example 1 were carefully cut into rectangular forms of 2 cm width and 6 cm length, seeded with fibroblast, and incubated for selected time points (Days 0, 15, 30, 60, 90). After incubation in vitro, samples were rinsed with distilled water before tensile testing to minimize the salt remnants from the cell culture media that would cause the samples to become stiff and break easily at the maximum load. Tensile testing was done at an extension rate of 5 mm/min. The gauge distance between the grippers was modified to 4 cm, and the width was modified to 2 cm.

The results showed that once the mesh implant is placed in a biological environment, the mesh implant losses about 20% of the original longitudinal tensile strength by the 10^{th} day and about 35% of the original transverse tensile strength by the 10^{th} day, which indicates a gradual biodegradability, as shown in FIG. 9. By 15 days, the mesh implant losses about 40% of its original longitudinal tensile strength and about 40% of its original transverse tensile strength. By 30 days, the mesh implant losses about 50% of its original longitudinal tensile strength and about 60% of its original transverse tensile strength. By 60 days, the mesh implant losses about 75% of its original longitudinal tensile strength and about 70% of its original transverse tensile strength. By 90 days, the mesh implant losses about 84% of its original longitudinal tensile strength and about 75% of its original transverse tensile strength. This loss of its original longitudinal tensile strength and original transverse tensile strength indicate that as the mesh implant when placed in a biological environment, the fibers gradually degrade which explains the loss of their mechanical strength.

### Example 8: Use of the Mesh Implant to Repair of Hernia Created in Animals

a) Mice Experiments

Adult male Balb/c mice were obtained from the animal care facility at our institution. Mice protocols were approved by the Institutional Animal Care and Use Committee of our institution. Mice were prepared for surgery as follows. Anesthesia was induced using inhaled isoflurane followed by ketamine-xylazine injection. The abdomens of the mice were then shaved and disinfected with chlorhexidine. Then, a 1-cm incision was done over the skin till the abdominal wall was reached. This was followed by creating a full-thickness abdominal wall 0.5×0.5 cm defect by retracting the abdominal wall by forceps and cutting it by sterile scissors. The induced hernia was repaired primarily using 5.0 Prolene interrupted sutures followed by application of 1×1 cm mesh implant of Example 1. The surgeon experienced no problems with securing the mesh implant to the body wall, and the mesh implant adapted to the shape of the abdominal wall. Animals were then monitored on a daily basis. None of the animals exhibited any signs of discomfort when moving or carrying on their daily activities that required applying pressure to the abdominal area. At one month following the surgery, mice were humanely sacrificed and the hernia site of each sacrificed mouse was autopsied.

b) Pig Experiments

Adult female pigs were obtained from the animal care facility at our institution. Pig protocols were approved by the Institutional Animal Care and Use Committee of our institution. Pigs were prepared for surgery as follows. Anesthesia was induced using inhaled isoflurane followed by ketamine-xylazine injection. The abdomens of the mice were then shaved and disinfected with chlorhexidine. Then, a 10-cm incision was done over the skin till the abdominal wall was reached. This was followed by creating a full-thickness abdominal wall 5×5 cm defect by retracting the abdominal wall by forceps and cutting it by sterile scissors. The induced hernia was repaired primarily using 5.0 Prolene interrupted sutures followed by application of 10×10 cm mesh implant of Example 1. The surgeon experienced no problems with securing the mesh implant to the body wall, and the mesh implant adapted to the shape of the abdominal wall. Animals were then monitored on a daily basis. None of the animals exhibited any signs of discomfort when moving or carrying on their daily activities that required applying pressure to the abdominal area. At one, two and six months following the surgery, pigs were humanely sacrificed and the hernia site of each sacrificed pig was autopsied.

### Example 9: Gross Examination of the Tissues from Repaired Hernia Site

Following the sacrifice of the mice and pigs of example 8a and 8b, respectively, samples were collected from the repaired hernia sites and examined grossly. At all tested time points, the results showed that there was good incorporation of the mesh implant with surrounding tissues, no signs of rejection, intact bowels after direct contact with mesh implant, and no evidence of hernia recurrence.

### Example 10: Histological Analysis of Pig Tissue Samples

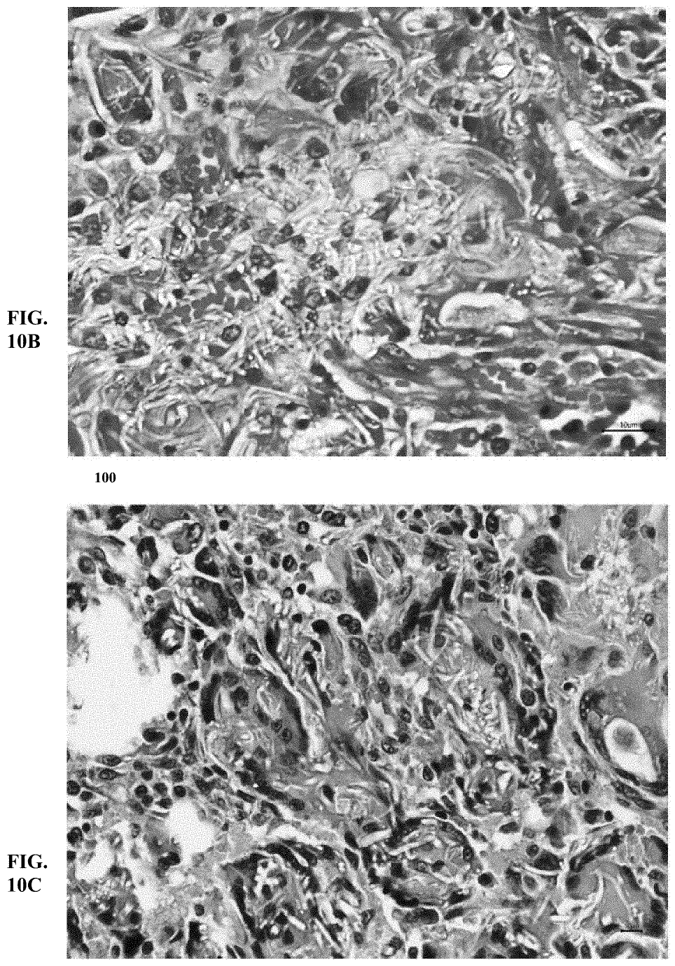

Following the sacrifice of the pigs of example 8b, samples were collected from the repaired hernia sites, fixed in 4% PFA, embedded in paraffin and sectioned. The sections were then stained with hematoxylin and eosin (H&E). The histological images of the collected tissues showed a steady presence of the mesh fibers in the tissues at one month (FIG. 10A), two months (FIG. 10B), and six months (FIG. 10C) following the hernia procedures. Nevertheless, the histological images of the collected tissues also showed a gradual decrease in the number of visible mesh nanofibers as time progressed from month one (FIG. 10A) to month two (FIG. 10B) to month six (FIG. 10C) following the hernia procedure. Therefore, following its placement on the hernia defect, the degradation of the mesh implant is gradual enough to allow the implant to stay in place for at least six months following the hernia procedure. Finally, the histological images of the collected tissues showed no signs of mesh implant rejection or tissue necrosis, and the mesh implant integrated well into the neighboring tissues and facilitated a healthy granulation process. These results indicate that the mesh implant facilitates wound healing at the repaired hernia site leading to a successful defect closure (FIGS. 10A, 10B & 10C).

### Example 11: Histological Analysis of Mouse Tissue Samples

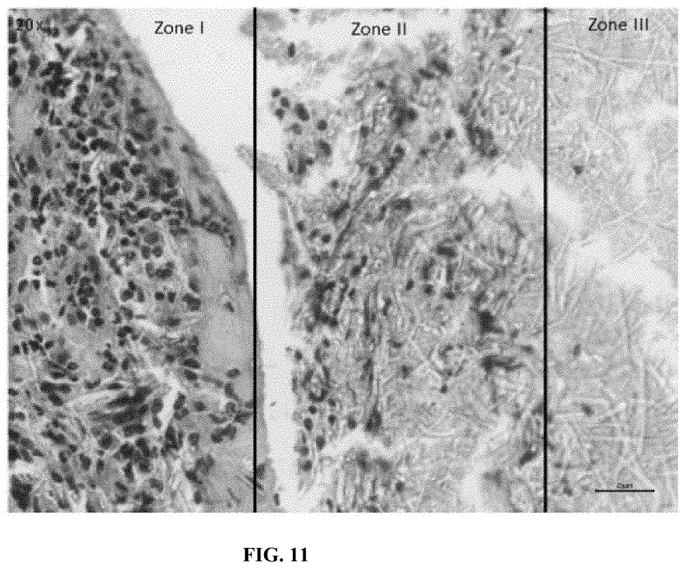

Following the sacrifice of the mice of example 8a, samples were collected from the repaired hernia sites, fixed in 4% PFA, embedded in paraffin and sectioned. The sections were then stained with hematoxylin and eosin (H&E). One histological image of a tissue sample collected one month following hernia repair captured cell infiltration of the mesh implant in progress (FIG. 11), where in one area, the mesh implant had been fully infiltrated by cells (Zone I), in another area the cell infiltration was in progress (Zone II), while a third area was cell-free as cell infiltration had not reached it yet (Zone III in FIG. 11). These results indicate that the mesh implant, through its highly fibrous 3D structure, promotes cell attachment and infiltration. On another note, the histological images of the collected tissues showed no signs of mesh implant rejection or tissue necrosis, and the mesh implant integrated well into the neighboring tissues and facilitated a healthy granulation process. These results represent an additional proof that that the mesh implant facilitates wound healing at the repaired hernia site leading to a successful defect closure (FIG. 11).

The examples above show that the mesh is used in tissue repair, namely hernia repair. The single-sheet mesh implant reduces the amount of foreign material that make up the mesh, which minimizes the risk of mesh rejection. The examples above support and enable a gradually biodegradable nature of the material that makes up the mesh guarantees that the mesh stays in place and supports the repaired site long enough until a proper scar tissue has built up, after which the mesh disappears from the body, therefore preventing persistent pain and irritability. The examples show and enable the mesh implants 3D design, nanofibrous structure, and high porosity of the mesh facilitate cell attachment, infiltration, and proliferation, all of which are necessary for scar tissue formation, mesh integration, wound healing, and proper defect closure. In one embodiment, the mesh is manufactured using electrospinning to give it its highly porous, nanofibrous, 3D structure. Moreover, electrospinning is an economic manufacturing technique with scalable productivity, which makes the mesh an affordable, yet profitable, product.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A mesh implant for hernia repair comprising: a single sheet of a gradually biodegradable synthetic polymer material; wherein the gradually biodegradable synthetic polymer is organized as a plurality of nanofibers randomly distributed and stacked to form a porous flat three-dimensional mesh; the porous flat three-dimensional mesh includes an original longitudinal tensile strength and an original transverse tensile strength; wherein the original longitudinal tensile strength ≥16N/cm and the original transverse tensile strength ≥16N/cm.

2. A mesh implant of claim 1, wherein the mesh implant losses about 20% of the original longitudinal tensile strength by the 10^{th} day once the mesh implant is placed in a biological environment, and the mesh implant losses about 35% of the original transverse tensile strength by the 10^{th} day once the mesh implant is placed in a biological environment through biodegradation.

3. A mesh implant according to claim 2, wherein the gradually biodegradable synthetic polymer material is a single polymer, a plurality of copolymers, a polymer blend, or an electrospun material.

4. A mesh implant according to claim 3, wherein said mesh is resistant to tissue adhesion and eventually degrades in the body after 6 months.

5. A mesh implant according to claim 4, wherein said mesh has a thickness ≥75 μm, wherein the nanofibers have a diameter ≥0.2 μm.

6. A mesh implant according to claim 5, wherein the porosity is an average of 70 pores/cm² and the pores include a diameter of ≥2 μm; and the mesh implant losing about 40% of its original longitudinal tensile strength and losing about 40% of its original transverse tensile strength by 15 days after the mesh implant is placed in the biological environment.

7. A method of using a mesh implant for hernia repair, further comprising placing a mesh implant on the uncovered fascia at the repaired hernia site and preventing hernia recurrence with the mesh implant, wherein the mesh implant includes an original longitudinal tensile strength and an original transverse tensile strength; and once the mesh implant is placed in at the hernia site, the mesh implant losses about 20% of the original longitudinal tensile strength by the 10^{th} day and about 35% of the original transverse tensile strength by the 10^{th} day through biodegradation; wherein the original longitudinal tensile strength ≥16N/cm and the original transverse tensile strength ≥16N/cm.

8. The method of claim 7, wherein the hernia repair is selected from the group consisting of: inguinal, femoral, umbilical, incisional, epigastric, and hiatal.

9. The method of claim 8, wherein the mesh implant does not cause connective tissue irritation that leads to mesh rejection and the mesh implant degrades gradually enough to stay in the tissue for at least six months.

10. The method of claim 9, wherein the mesh implant supports the repaired hernia site until a scar tissue is formed.

11. The method of claim 10, wherein said mesh implant does not cause long-term pain, irritation, or restricted mobility for the subject.

12. The method of claim 11, wherein the mesh implant mimics the structure of the extracellular matrix and facilitates cell attachment, cell infiltration, cell proliferation, and cell differentiation.

13. The method of claim 12, wherein white blood cells and fibroblasts infiltrate, attach to, and grow on the nanofibers of said mesh implant.

14. The method of claim 13, wherein said mesh implant integrates into the neighboring tissues at the repaired hernia site and the mesh implant facilitates proper wound healing and defect closure.

15. The method of claim 14, further comprising growing isolated differentiable cells on the mesh implant as a scaffold.

16. A mesh implant according to claim 15, wherein said isolated differentiable cells include, but are not limited to, the stem cells or progenitor cells of the blood, cartilage, bones, skin, and nerves.

17. A mesh implant according to claim 16, further comprising using the mesh implant in the repair of tissue injury including wound healing, repair of injuries to the bone, nerves, or skin, and treatment of burn injuries.

18. A method of making a mesh implant, comprising electrospinning a mesh implant to achieve a porous nanofibrous three-dimensional structure, wherein the porous nanofibrous three-dimensional structure includes an original longitudinal tensile strength ≥16N/cm and an original transverse tensile strength ≥16N/cm the mesh implant, wherein the porous nanofibrous three-dimensional structure losses about 20% of the original longitudinal tensile strength by the 10^{th} day and about 35% of the original transverse tensile strength by the 10^{th} day through biodegradation.

19. The method according to claim 18, wherein the electrospinning includes parameters of applied voltage, PLDL initial concentration, polymer solution feed rate, tip to collector distance, and collector rotational speed; and selecting the parameters such that the manufactured mesh has maximized thickness of 200 μm, a porosity of 70 pores/cm², and an average pore size of ≥2 μm.

20. The method according to claim 19, further comprising electrospinning under a low applied voltage and a low collector rotational speed.
